(19) United States
(12) Patent Application Publication
(10) Pub. No.: US 2020/0206520 A1
(43) Pub. Date: Jul. 2, 2020

(54) A METHOD OF DETERMINING SUITABILITY OF ADMINSTERING DEFIBRILLATOR ELECTRIC SHOCK AND A DEVICE THEREOF

(71) Applicants: Chung Wah SIU, Hong Kong (CN); HEARTISANS LIMITED, Hong Kong (CN)

(72) Inventors: Hin Wai LUI, Hong Kong (CN); Chung Wah SIU, Hong Kong (CN)

(21) Appl. No.: 15/998,898

(22) PCT Filed: Feb. 16, 2016
(86) PCT No.: PCT/CN2016/073876

§ 371 Date: Aug. 16, 2018

(51) Int. Cl. A61N 1/39; A61B 5/026

(52) U.S. Cl. CPC A61N 1/3987; A61N 1/3956; A61B 5/0261

(57) ABSTRACT

A method of determining whether rapid pulse indicative of ventricular tachycardia (VT) or ventricular fibrillation (VF) requires electric shock to defibrillate the rapid pulse using blood flow as a robust indicator of blood pressure stability. Preferably, the blood flow monitor is an optical sensor capable of monitoring blood flow change the moment VT or VF has begun its onset.

### FIELD OF THE INVENTION

The current invention relates to the field of cardioverter defibrillators.

### BACKGROUND

Ventricular tachycardia (VT) is an abnormally rapid heart rate that arises from improper electrical activity in the bottom chambers (ventricles) of the heart. During VT, the ventricles contract in a rapid, unsynchronized way. That is, the ventricles “fibrillate” instead of beat rhythmically at a healthy pace. As a result, the heart may pump little or no blood. This is a potentially life-threatening cardio arrhythmia because it can cause low blood pressure and may lead to ventricular fibrillation (VF), sudden cardiac arrest (SCA) and even death.

VT is sometimes defined as a heart rate of more than 100 beats per minute with at least three irregular pulses in a row. VF is a more severe condition defined by an even faster heart rate. For convenience, hereinafter, mention of VT shall be taken to include VF where it is reasonable to deem so.

People who have suffered cardiac arrest or have suffered VT before would require continuous monitoring for sudden episodes of VT. One way of providing twenty-four hour monitoring of a person for VT is to implant an ICD (implantable cardioverter defibrillator) into the person.

An ICD is an electronic device that constantly monitors a person's heart rhythm and is usually inserted surgically under the skin in the left upper chest, or abdomen of the person. Typically, an ICD comprises a generator and one or more electrode leads. The generator contains a computer chip or circuitry with a RAM memory, software, a capacitor and a long lasting battery. The electrode lead is connected to the generator and passes through a vein to contact a selected heart chamber, which may be the ventricle, the right atrium or the coronary sinus. The simplest ICD has a single lead whereas more complex ones have two or three leads, each lead connecting to a different part of the heart. Each lead comprises a pair of electrodes. The electrodes are used to monitor the heart rhythm on one hand and to deliver a therapeutic electrical shock on the other. When an ICD detects a very rapid, abnormal heart rate, it delivers an electrical shock to the heart chamber through the lead, to defibrillate the rapid heart rate, i.e. restores normal heart rhythm. Typically, the shock administered by ICDs is up to 40 Joules. This is lower than the level administered by external, non-implanted defibrillators which are operated by paramedic personnel, at about 100-360 Joules. However, to provide suitable therapy to different types of arrhythmic heart rate, it has been proposed that the ICDs administer a variety of electric shock types. For example, when the heart rate is too fast, a series of small electrical impulses may be delivered. However, when the heart rate is dangerously fast, a high-energy shock is delivered.

Typically, the lead obtains in real time the electrocardiographic (ECG) signal of the heart rhythm, which are analysed to determine the heart rate. The heart rate is typically obtained from the duration of each cardiac cycle on a heartbeat-to-heartbeat basis. Identifying VT from normal heart rhythm in this way is known as heart rate discrimination. However, not all racing heart rhythm indicates VT.

To more accurately determine whether there is an occurrence of VT when fast heart rate is detected, many ICDs now include in the VT detection process an evaluation of the morphology of the pulse, known as morphology discrimination. This is simply a method of correlating a template of a single, normal pulse signal with a current sample of the person's pulse. If the morphology of the person's current pulse correlates with the template, the fast heart rate is not considered indicative of VT and no electric shock will be administered. If the pulse does not correlated with the template, the ICD will determine that there is VT and deliver electric shock to defibrillate the fast heart rate.

Despite such complex signal processing algorithms to determine VT accurately and despite a variety of strategies to determine whether therapy is appropriate, there have been incidents of inappropriate therapy, i.e. therapy has been applied to people when their condition does not actually require the therapy. For example, in some cases, despite a fast pulse and abnormal pulse morphology, the person's heart manages to pump blood somewhat normally. This kind of VT is known as hemodynamically stable VT, which does not affect blood flow in the patient. Hence, the heart of this person does not need any electric shock to help it revert to normal function. Administering electric shock to the heart in this case does more harm than benefit. Unfortunately, the algorithms currently employed by ICDs are unable to distinguish hemodynamically stable VT (referred to as stable VT hereinafter) from hemodynamically unstable VT (referred to as unstable VT hereinafter).

If a non-implanted, external cardioverter defibrillator which is operated by paramedic personnel manually is used, the current strategy is to let the person suffer VT for a while to see if the person shows signs of cardiac arrest. If no cardiac arrest is observed, it means that the VT is hemodynamically stable and no electric shock therapy is required. However, if the person starts to show signs of cardiac arrest, an electric shock will be administered by placing the electrodes of the external cardioverter defibrillator on his chest. In some ICDs, a similar strategy is used, wherein a compulsory waiting period is required on detection of fast heart rate before electric shock therapy may be administered. This is a dangerous strategy as it allows precious time which could have been used for life-saving defibrillation slip away while the person is being observed.

Therefore, one of the key challenges in proper use of cardioverter defibrillators is the inability of cardioverter defibrillators to differentiate between stable VT and non-stable VT.

Accordingly, it is desirable to provide a method or device for distinguishing stable VT from unstable VT, possibly increasing the likelihood of applying appropriate therapy to treat VT.

### STATEMENT OF INVENTION

In a first aspect, the invention proposes a method of determining suitability of administering defibrillator electric shock, comprising the steps of: detecting ventricular tachycardia (VT) in a person; obtaining an indication of the blood pressure of the person; if the blood pressure is below a pre-determined threshold, determining that administering a defibrillator electric shock is appropriate.

For convenience, hereinafter, mention of VT shall be taken to include VF where it is reasonable to deem so.

Optionally, the threshold is a fixed value to be applied on persons on whom the method is applied, and the value may be determined by studying the typical rate of blood flow in most people. In this case, the threshold is applied regardless of each individual user.

Alternatively, the threshold is a value which is read dynamically when there is an onset of VT. Using such a dynamically determined threshold removes false alarms which can happen if the threshold is a fixed value, i.e. as blood pressure fluctuates naturally and varies from person to person, if VT happens during a moment of natural but relatively lower blood pressure, a false alarm that the VT has caused low blood pressure may be raised.

In yet another alternative, the threshold is the blood pressure of the person taken just before the onset of the cardio arrhythmia.

Stable VT and non-stable VT are distinguished apart based on whether the heart is able to pump blood during an episode of VT. Typically, non-stable VT may be determined by observing a drop in blood pressure concurrent with the onset of a rapid heart rhythm. Accordingly, by requiring a drop in blood pressure to be observed before defibrillation is administered, the chance of applying inappropriate therapy to a person suffering from stable VT may be avoided.

Preferably, the method further comprises the step of: assessing blood flow in the person's skin; wherein a drop in skin blood flow or skin blood level is taken to indicate a corresponding drop in blood pressure. More preferably, the method further comprises the step of: assessing skin blood flow by monitoring light reflected off the skin.

Skin blood flow is indicative of blood pressure and can be assessed quickly, and monitoring skin blood flow is therefore a robust, rugged but reliable method for assessing blood pressure.

However, blood pressure detection using a clinical device such as a sphygmomanometer requires time and a skilled operator. If a sphygmomanometer is used to obtain the blood pressure of a person suffering VT, the sphygmomanometer must be allowed to take some time to complete its measurement process. Therefore, reading a person's blood pressure by a sphygmomanometer when he is suffering VT wastes precious time which could be used to save this life if his episode of VT is unstable. Furthermore, a sphygmomanometer reading is only useful for measuring blood pressure in a moment and is unable to track dynamically changing or dropping blood pressure. Advantageously, invention overcomes these problems associated with using a sphygmomanometer by assessing skin blood flow using optical sensing to detect unstable VT, such as a PPG (photoplethysmocharty).

A PPG sensor is able to determine dynamic change in blood flow within the top skin layers, and monitoring blood flow is sufficient for the purpose of determining qualitatively whether there is significant blood pressure drop. This is enough for the purpose of establishing whether the episode of VT is stable or not, and a precise quantifying of the exact amount of blood pressure drop is not necessary. This leads to a further advantage that blood pressure monitoring for the purpose of establishing whether a VT episode is stable or not may be done by devices more robust and rugged than a sphygmomanometer. Ruggedness in a device's design is an important quality, which possibly allows the device to be carried or worn by a person throughout the day, without significant risk of the device malfunctioning easily. In particular, a light based blood flow assessment device can be rugged and robust, capable of being used while the person is engaged in sports or other daily activities, and also capable of being manufactured cheaply. In contrast, a clinical sphygmomanometer cannot be worn be a person throughout the day and is easily damaged when carried about

Typically, the person is implanted with a cardioverter defibrillator comprising a wireless transceiver, and the person is wearing a skin blood flow monitor for the assessment of skin blood flow, and the method further comprising the step of: when the cardioverter defibrillator detects ventricular tachycardia, the cardioverter defibrillator instructing the skin blood flow monitor to commence the assessment of skin blood flow.

Alternatively, the method comprises the steps of: providing a cardioverter defibrillator for the administration of defibrillator electric shock, providing a skin blood flow monitor for the assessment of skin blood flow, the cardioverter defibrillator obtaining skin blood flow information from the skin blood flow monitor before delivering a defibrillator electric shock.

Preferably, the method is implemented by using a blood flow monitor as a form of wearable electronics, such as a wristband-like device. Wearing the device on the wrist can be advantageous as there is relatively little muscular movement in that part of the body and an optical system can be placed on the skin at the wrist snugly for accurate reading. Advantageously, a wearable device does not require implantation into the person's body, and is therefore a non-invasive way of improving existing ICD performance. In addition, existing ICDs are provided with a wireless transceiver, and the firmware in existing ICDs may be re-programmed to work with the proposed wearable blood flow monitor, without need to replace implanted ICDs while improving their performance.

In a second aspect, the invention proposes a method of determining hemodynamically unstable cardio arrhythmia, comprising the step of: detecting cardio arrhythmia in a person; irradiating the person's skin with a source of light; monitoring change in skin blood flow by observing a change in the amount of reflected light detected; and determining hemodynamically unstable cardio arrhythmia if the cardio arrhythmia is accompanied by a drop in skin blood flow below a pre-determined threshold.

On one hand, the threshold is the person's skin blood flow just when ventricular tachycardia is detected. Alternatively, the threshold is the person's skin blood flow before ventricular tachycardia is detected. In yet another alternative, the threshold is a fixed value which applies regardless of each individual user.

In a third aspect, the invention proposes a cardioverter defibrillator configured to require information from a blood pressure monitor before the cardioverter defibrillator is capable of delivering a therapeutic electrical shock.

Preferably, the blood pressure monitor is in the form of a skin blood flow monitor.

Optionally, the cardioverter defibrillator is an implantable cardioverter defibrillator comprising: a wireless receiver configured to receive the indication from the skin blood flow monitor wirelessly.

In a fourth aspect, the invention proposes a portable blood pressure monitor suitable for determining suitability of administering defibrillator electric shock, comprising: a light source for irradiating the person's skin; an optical sensor for detecting light reflected from the skin; a microcontroller and memory module configured for assessing skin blood flow from the intensity of light reflected from the skin; and a communication port for communicating to a cardioverter defibrillator information related to skin blood flow.

Optionally, the communication port is a wireless communication port.

Optionally, the cardioverter defibrillator is an implantable cardioverter defibrillator having a corresponding wireless communication port.

### BRIEF DESCRIPTION OF DRAWINGS

It will be convenient to further describe the present invention with respect to the accompanying drawings that illustrate possible arrangements of the invention, in which like integers refer to like parts. Other embodiments of the invention are possible, and consequently the particularity of the accompanying drawings is not to be understood as superseding the generality of the preceding description of the invention.

FIG. 1 shows an embodiment of the invention;

FIG. 2 shows the embodiment of FIG. 1 in a different position;

FIG. 3 is a schematic illustration of the components of the embodiment of FIG. 1

FIG. 4 is the heart rhythm read by the embodiment of FIG. 1 when blood flow is stable;

FIG. 5 is the heart rhythm read by the embodiment of FIG. 1 when blood flow is dropping;

FIG. 6 is heart rhythm as observed by ECG;

FIG. 7 illustrates the embodiment of FIG. 1 worn by a user. and

FIG. 8 is a flowchart showing how the embodiment of FIG. 1 operates with a cardioverter defibrillator.

### DESCRIPTION OF PREFERRED EMBODIMENTS

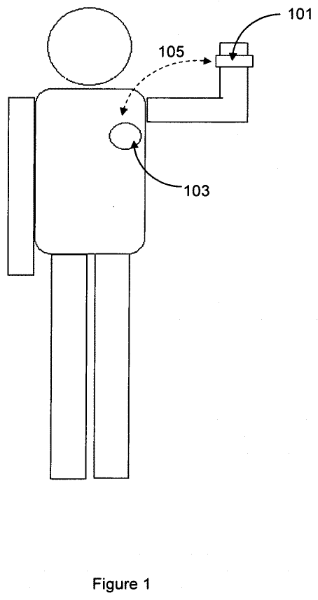

FIG. 1 schematically illustrates a person implanted with an implantable cardioverter defibrillator (ICD) 103. The ICD 103 is in wireless communication (illustrated by the broken line arrow 105) with a blood flow monitor 101. The blood flow monitor 101 is worn on the wrist of the person. The ICD cooperates with the blood flow monitor 101 to determine whether electric shock defibrillation therapy is required in the event that ventricular tachycardia (VT) is detected in the person.

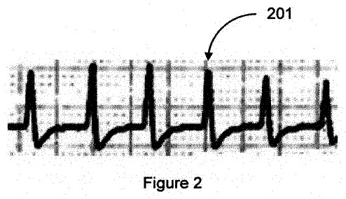

FIG. 2 shows a series of pulse signals 201 in the form of electrocardiogram detected by the ICD 103 from the person's heart. If the heart rate is determined to be abnormally fast by the ICD, such as being greater than 100 beats per minute, the morphology of a pre-determined number of pulses is checked by the ICD to see if an abnormal pulse shape is detectable. If the heart rate is fast and the pulses exhibit abnormal morphology, the ICD determines that it is VT. The ICD immediately communicates with the blood flow monitor 101 to check whether there is a concurrent drop in blood level in the person's skin, i.e. by checking whether blood flow in skin has slowed down significantly.

If the blood pumping function of the heart is stable, the person does not suffer any drop in blood pressure, and blood flow in the skin of the person will also remain stable. Therefore, if no significant change in the skin blood flow is detected, it means that the VT is hemodynamically stable and does not require electric shock to be defibrillated.

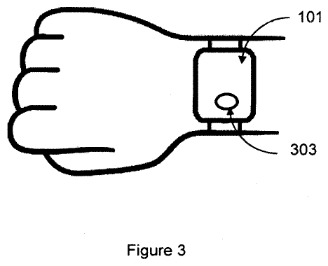

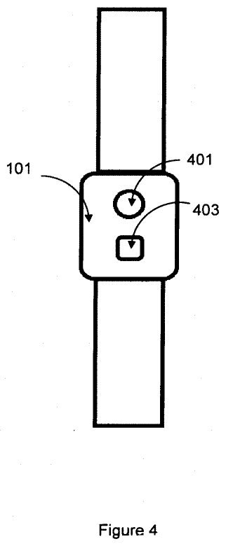

FIG. 3 shows a blood flow monitor 101 strapped to the wrist of a person. FIG. 4 provides a view of the underside of the blood flow monitor 101. On the underside of the blood flow monitor 101 is a PPG (photoplethysmocharty) sensor. A PPG sensor uses light-based technology to sense blood flow as provided by the heart's pumping action. Typically, a PPG sensor comprises at least one light source 401 such as an LED (light emitting diode) and one corresponding optical sensor 403.

Making the blood flow monitor 101 into the form of a wristband as shown in FIG. 3 provides certain advantages. The wrist is the location on the patient's body that can provide good reading of the blood flow, and is the least intrusive to the patient's everyday life. However, other locations are possible, such as the finger in which case the blood flow monitor 101 can be made into a finger sheath or a ring for monitoring blood flow change in the finger.

The blood flow monitor 101 is designed to be worn such that the light source 401 and the optical sensor 403 are placed snugly against the skin, in order to prevent ambient light from causing too much noise signals in the optical sensor 403. The light source 401 transmits light into the person's skin, and the light is diffused and reflected by the surface of the skin towards the optical sensor 403. ‘Reflection’ is taken here to include the case wherein light penetrates beneath the skin surface but are diffused or rebounded back by the top layers of skin and tissue towards the optical sensor 403. The reflected light has a varying intensity which fluctuates in accordance with the pulsation of blood in the person's skin. In this way, the optical sensor 403 is able to monitor blood flow in the person's skin.

A PPG sensor is small and only requires a single point of contact on a person's body. Therefore, using a PPG sensor allows a small blood flow monitoring device to be made into a convenient and portable form, such as the wrist worn configuration shown in FIG. 1, to be deployed on and worn by a person around the clock daily. Furthermore, PPG sensors are relatively cheap and sufficiently rugged for long term, daily use.

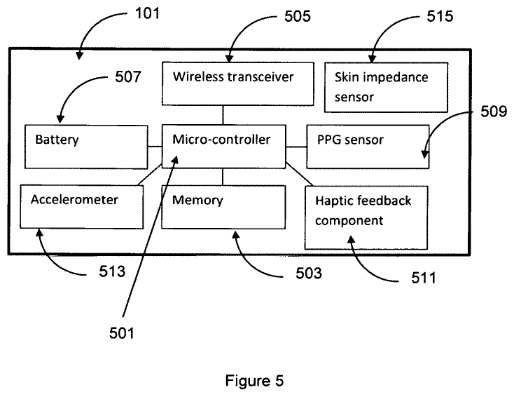

FIG. 5 is a schematic diagram of one possible internal structure of the blood flow monitor 101. Typically, the blood flow monitor 101 comprises a microcontroller 501 and a memory 503. The microcontroller 501 operates the PPG sensor 509. That is, the microcontroller 501 operates the light source 401 to illuminate the person's skin and operates the optical sensor 403 to detect the intensity of the light reflected from the person's skin. The memory 503 contains an algorithm for assessing the blood flow of the person from the reflected light.

Furthermore, the blood flow monitor 101 comprises a wireless transceiver 505 for communicating information on detected blood flow. The wireless technology used for communication between the ICD 103 and the blood flow monitor 101 may be Bluetooth™, Wi-Fi, Near Field Communication, infrared communication or any other suitable wireless communication protocol. However, the wireless communication protocol is preferably Bluetooth Low Energy. The top face of the blood flow monitor 101 as shown in FIG. 3 comprises a button 303 for initiating Bluetooth synchronisation with an ICD.

Furthermore, a replaceable and rechargeable battery 507 is provided to supply power to all the components in the blood flow monitor 101. The battery 507 is preferably a rechargeable and replaceable one, as it allows the person to swap the battery 507 quickly at any time, such that there is no need to wait for the battery 507 to charge up. This provides the advantage that the person may benefit from almost seamless, continual monitoring of his heart for unstable VT.

The blood flow monitor 101 preferably comprises an accelerometer 513 for detecting movements of the person wearing it. If the accelerometer detects no movements for an extended period of time, the microcontroller 501 will consider that there blood flow monitor 101 is not being worn and will go into sleep mode to conserve battery power.

Optionally, the blood flow monitor 101 comprises a skin impedance sensor 515 (not illustrated in FIG. 1) positioned on the underside of the blood flow monitor 101, adjacent the light source 401 and the optical sensor 403. A skin impedance sensor 515 measures impedance or conductance of skin surface. Impedance of skin is different from that of air. Therefore, if the skin impedance sensor 515 is in contact with the skin of the person, certain impedance should be measured. If there is a small gap between the person's skin and the optical sensor 403, there will also be a corresponding small gap between the skin impedance sensor 515 and the skin, and the skin impedance sensor 515 will not detect impedance typical of skin but will detect impedance somewhat typical of air. In this way, the skin impedance sensor 515 is useable to determine whether the light source 401 and the optical sensor 403 have been placed in sufficiently tight contact with the skin in order for the optical sensor 403 to read heart beat properly, reducing the possibility of ambient light affecting the reading of the optical sensor 403.

Preferably, if the skin impedance sensor 515 determines that the light source 401 and the optical sensor 403 are not in contact with the skin, data read by the optical sensor 403 is rejected and not taken to assess the blood flow.

Preferably, the blood flow monitor 101 is able to alert the person that the light source 401 and the optical sensor 403 are not placed tightly enough against the skin, such as by issuing a series of haptic signals in a specific rhythm. For this purpose, a haptic feedback component 511 is provided in the blood flow monitor 101.

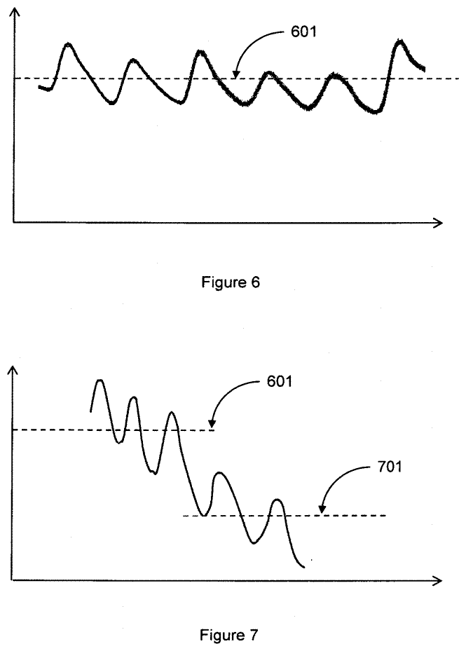

In use, the PPG sensor 509 assesses the person's blood flow in real time. FIG. 6 is a chart which shows how light reflected from the skin of a person wearing the blood flow monitor 101 represents the pulse under normal condition. The vertical axis of the chart represents intensity of the reflected light while the horizontal axis represents time. In preferred embodiments, the light emitted by the light source 401 has been selected to have a wavelength which is reflected by blood and not absorbed. Therefore, the more blood is in the skin, the stronger the reflected light intensity. The intensity of the reflected light detected by the optical sensor 403 pulsates in tandem with the pulsation of blood in the skin. Normally, the intensity of the reflected light fluctuates about a mean value illustrated by the broken line 601, which correlates to the average or mean light absorption in the person's skin.

FIG. 7 shows what the optical sensor detects when a person's heart is unable pump blood through the body, such as in the case of unstable VT. Blood level in the person's skin layer drops as blood fails to reach the skin. As a result, there is less blood in the skin reflecting light to the optical sensor 403. Accordingly, and the intensity of the reflected light is seen dropping downwardly. This is the reason why a person turns pale visibly when suffering a drop in blood pressure. In other words, the PPG sensor 509 is also able to detect this overall drop of blood in the skin as an indication of a drop in blood pressure. The lower broken line 501 in FIG. 5 illustrates a threshold 501 wherein, if the reflected light intensity detected by the optical sensor 403 drops below this threshold, the microcontroller 501 in the blood flow monitor 101 will determine that the skin is under-supplied with blood, representing a drop in blood pressure. Typically, the threshold 501 is not used to monitor every fluctuation in detected light intensity but used to monitor a moving average of the detected light intensity, in order to prevent false alarms. The length or period of the moving average may be determined in each product made based on the embodiment by the manufacturer and is a detail which is not of particular concern here.

Preferably, the micro-controller 901 in the blood flow monitor 101 is capable of performing signal processing on the detected light to allow removal of noise.

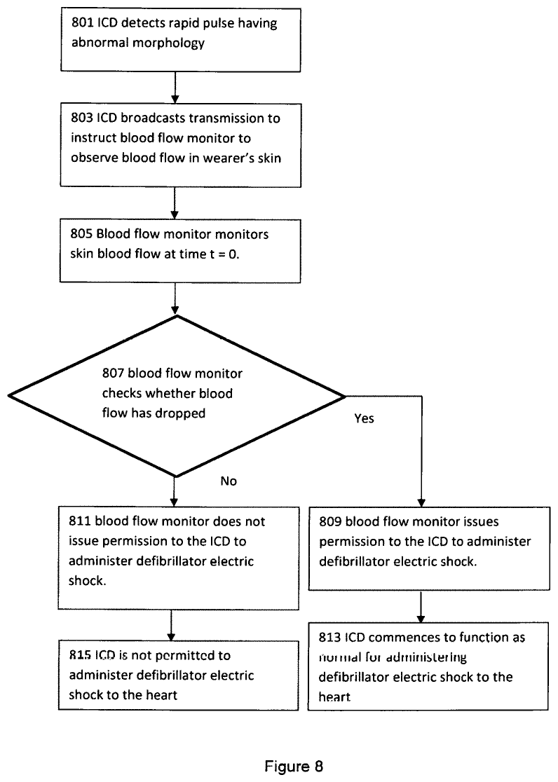

FIG. 8 is a flowchart showing possible steps of how the ICD 103 cooperate with the blood flow monitor 101 to determine if an episode of VT is stable or unstable. In step 801, when the ICD 103 determines that it has detected VT (by any conventional ICD design and method), the ICD 103 initiates communication with the blood flow monitor 101, at step 803. The blood flow monitor 101 is always actively listening for such instruction from the ICD 103 despite being in a sleep mode normally. When the blood flow monitor 101 detects the an instruction broadcasted from the ICD, the blood flow monitor 101 awakes and begins monitoring fine or minute changes blood level in the person's superficial layers of the person's skin, at step 805. Optionally, the blood level which the blood flow monitor 101 observes from reflected light when it awakes is taken as the reference blood flow, i.e. as time=0. Subsequently, blood level change is measured against the reference blood level, at step 807. If the person's blood level has changed more than a predetermined threshold, such as if the blood flow monitor 101 detects that the person's blood level has dropped significantly from the reference blood level within a specific period of time, the skin monitor will record a positive indication that there has been a drop in the person's blood pressure. How much drop in blood level or blood flow is considered significant may depend on manufacturer's preferred settings. The blood flow monitor 101 then issues permission to the ICD, at step 809, allowing the ICD 103 to deliver defibrillating electric shock as required, at step 813. Alternatively, instead of using the blood level which the blood flow monitor 101 observes when it awakes is taken as a reference blood flow, i.e. as time=0, an absolute threshold level may be set, as discussed for FIG. 7.

If the person's blood level has not changed significantly, the blood flow monitor 101 will not record a positive indication that there has been a significant drop in the person's blood pressure. In this case, the blood flow monitor 101 does not issue permission to the ICD, at step 811 and step 815, to deliver any defibrillating electric shock.

Therefore, the embodiment described relates to an ICD 103 which is able to communicate with another sensor 101, and the other sensor 101 is able to provide an indication of the blood pressure of the person in real time to the ICD, in order to allow the ICD 103 to differentiate between stable VT and non-stable VT. Advantageously, the sensor 101 being wearable externally does not require any surgical routine to be used on the person. Also, the embodiments described comprises a method of determining suitability of administering defibrillator electric shock comprising the steps of: detecting possible VT in a person, obtaining an indication of the blood flow of the person, if the blood flow of the person has dropped below a pre-determined threshold, determining that defibrillator electric shock therapy to the person's heart is suitable, and if the blood flow of the person has not dropped below a pre-determined threshold, determining that defibrillator electric shock therapy to the person's heart is not suitable.

While there has been described in the foregoing description preferred embodiments of the present invention, it will be understood by those skilled in the technology concerned that many variations or modifications in details of design, construction or operation may be made without departing from the scope of the present invention as claimed.

For example, where VT has been mentioned, the skilled man understands that the embodiments describe may apply to VF and other forms of abnormally rapid heart rate.

For example, in a variation of the embodiment, the blood flow monitor 101 continues to observe the blood flow of the person even when the VT has been determined as unstable and electric shock has been administered (not illustrated in flowchart). In this option, only a consistently low blood pressure will continue to allow the ICD 103 to administer further electric shocks. Once significant amount of blood flow returns in the skin, which shows that there is a rise in blood pressure, the blood flow monitor 101 will disallow the ICD 103 from administering further electric shock. Conversely, if the blood flow monitor 101 determines that there is no blood pressure drop in an episode of stable VT, the blood flow monitor 101 continues to monitor the blood flow in case the VT turns unstable and the blood pressure drops.

In a further variation of the embodiment, determination of whether the VT is stable or unstable is done as a one-off determination. When the blood flow monitor 101 receives instruction from the ICD 103 and commences to monitor blood flow change, and if the blood pressure is deemed to have dropped and unstable VT has been determined, the blood flow monitor 101 will provide one-off permission to the ICD 103 to administer as many electric shocks as it deems necessary. The ICD 103 does not consult the blood flow monitor 101 again. If the blood flow monitor 101 determines that there is no blood pressure drop and therefore the VT is stable, the blood flow monitor 101 simply disallows the ICD 103 from administering electric shock at all, and does not continue to monitor the blood flow for the current episode of VT while it lasts.

In yet a further variation of the embodiment, the blood flow monitor 101 does not actively permit or disallow the ICD 103 to administer electric shock. Instead, the blood flow monitor 101 only transmits information on blood flow change to the ICD, and it is the microcontroller within the ICD 103 which determines whether the electric shock should be administered. This variation of the embodiment provides the possible advantage that the ICD 103 is able to override any indication of a lack of blood pressure drop by the blood flow monitor 101, as decision is not made in the blood flow monitor 101. For example, if the pulse morphology, rhythm or frequency has exceeded a certain threshold and is deemed highly dangerous, the ICD 103 may administer the electric shock regardless of blood flow change of blood pressure information.

In yet another variation of the embodiment, the wavelength of the light may be selected such that light transmission or light absorption is used to detect change in blood flow instead of light reflection. For example, light transmitted through the person's finger may be used to determine if blood pressure has dropped. However, light transmission requires more energy which is less suitable for use in a twenty-four hour monitoring device.

In yet another variation of the embodiment, a suitably configured sphygmomanometer may nevertheless be used instead of the blood flow monitor 101. However, a clinical sphygmomanometer requires a long time to determine the blood pressure of the person. By the time a sphygmomanometer gives its reading, the person may have been endangered irreversibly, as precious time may be wasted for delivering the potentially life-saving electric shock.

While blood flow is one of the quickest observable changes when a person's blood pressure drops, there are other indicators which may be used instead. Cruder and less effective embodiments may monitor skin temperature, or blood oxygen level (by using infrared transmission through a finger) as an indicator of blood pressure drop. However, blood flow change tends to be observable well before any change these other indicators may be detected.

Optionally, infrared components may be monitored instead of visible light components to determine if blood flow in the skin has dropped or not.

Although an ICD 103 has been described so far, the described methods and features may also apply to external cardioverter defibrillator. In this case, the blood flow monitor 101 can be in communication with the external cardioverter defibrillator wireless or through a communication cable. A microcontroller in the external cardioverter defibrillator receiving information from the blood flow monitor 101 may make a decision whether administration of electric shock is appropriate. If the external cardioverter defibrillator is capable of making the decision, the external cardioverter defibrillator can be designed to be unable to charge up until the VT is confirmed to be unstable. Alternatively, the decision may be made by a human operator, and the blood pressure information is provided merely to the operator through an information display screen.

Similarly, although an ICD 103 has been described, the described technology and methods may also apply to Wearable CD (WCD) which are not implanted into the person, but are worn on his body. Similarly, although an ICD 103 has been described, the described technology and methods may also apply to external defibrillators which are used by paramedics. The cardioverter defibrillators in this case is required to communicate with a blood pressure monitor before the electric shock may be administered.

Although the embodiments described cardioverter defibrillators applied to a ‘person’, it is intended that ‘person’ refers to animals as well.

Although the embodiments have been generally described to detect blood flow using reflected light, the skilled man will understand that variations such as measuring the amount of absorbed light, transmitted light, scattered light are envisaged as within the scope of the invention.

1. A method of determining suitability of administering defibrillator electric shock, comprising the steps of:
detecting ventricular tachycardia in a person;
obtaining an indication of the blood pressure of the person;
if the blood pressure is below a pre-determined threshold, determining that administering a defibrillator electric shock is appropriate.

2. A method of determining suitability of administering defibrillator electric shock as claimed in claim 1, wherein
the threshold is pre-determined by obtaining the blood pressure when ventricular tachycardia is detected.

3. A method of determining suitability of administering defibrillator electric shock as claimed in claim 1, wherein
the threshold is pre-determined by obtaining the blood pressure before ventricular tachycardia is detected.

4. A method of determining suitability of administering defibrillator electric shock as claimed in claim 1, further comprising the step of:
assessing blood flow in the person; wherein
a drop in blood flow is taken to indicate a corresponding drop in blood pressure.

5. A method of determining suitability of administering defibrillator electric shock as claimed in claim 4, further comprising the step of:
assessing skin blood flow by monitoring light reflected off or absorbed by the skin.

6. A method of determining suitability of administering defibrillator electric shock as claimed in claim 5, wherein
the person is implanted with a cardioverter defibrillator comprising a wireless transceiver; and
the person is wearing a skin blood flow monitor for the assessment of skin blood flow;
further comprising the step of:
when the cardioverter defibrillator detects ventricular tachycardia, the cardioverter defibrillator instructing the skin blood flow monitor to commence the assessment of skin blood flow.

7. A method of determining suitability of administering defibrillator electric shock as claimed in claim 6, wherein
the cardioverter defibrillator instructs the skin blood flow monitor to assess skin blood flow after an episode of ventricular tachycardia.

8. A method of determining suitability of administering defibrillator electric shock as claimed in claim 5, further comprising the steps of:
providing a cardioverter defibrillator for the administration of defibrillator electric shock;
providing a skin blood flow monitor for the assessment of skin blood flow;
the cardioverter defibrillator obtaining skin blood flow information from the skin blood flow monitor before delivering a defibrillator electric shock.

9. A method of determining hemodynamically unstable cardio arrhythmia, comprising the step of:
detecting cardio arrhythmia in a person;
irradiating the person's skin with a source of light;
monitoring change in skin blood flow by observing a change in the amount of reflected or absorbed light detected; and
determining hemodynamically unstable cardio arrhythmia if the cardio arrhythmia is accompanied by a drop in skin blood flow below a pre-determined threshold.

10. A method of determining hemodynamically unstable cardio arrhythmia, as claimed in claim 9, wherein
the threshold is the person's skin blood flow when ventricular tachycardia is detected.

11. A method of determining hemodynamically unstable cardio arrhythmia, as claimed in claim 9, wherein
the threshold is the person's skin blood flow before ventricular tachycardia is detected.

12. A cardioverter defibrillator configured to require information from a blood pressure monitor before the cardioverter defibrillator is capable of delivering a therapeutic electrical shock.

13. A cardioverter defibrillator as claimed in claim 12 wherein the blood pressure monitor is in the form of a skin blood flow monitor.

14. A cardioverter defibrillator as claimed in claim 13 wherein the cardioverter defibrillator is an implantable cardioverter defibrillator comprising:
a wireless receiver configured to receive the indication from the skin blood flow monitor wirelessly.

15. A portable blood pressure monitor suitable for determining suitability of administering defibrillator electric shock, comprising:
a light source for irradiating the person's skin;
an optical sensor for detecting light reflected from the skin;
a microcontroller and memory module configured for assessing skin blood flow from the intensity of light reflected from the skin; and
a communication port for communicating to a cardioverter defibrillator information related to skin blood flow .

16. A portable blood pressure change monitor as claimed in claim 15 wherein the communication port is a wireless communication port.

17. A portable blood pressure change monitor as claimed in claim 15 wherein the cardioverter defibrillator is an implantable cardioverter defibrillator having a corresponding wireless communication port.
